(19) United States
(12) Patent Application Publication
(10) Pub. No.: US 2018/0028414 A1
(43) Pub. Date: Feb. 1, 2018

(54) METHOD FOR PRODUCING SOLID POWDER COSMETIC MATERIAL AND SOLID POWDER COSMETIC MATERIAL

(71) Applicant: TOKIWA CORPORATION, Nakatsugawa-shi (JP)

(72) Inventor: Yutaka MOTOKI, Kawaguchi-shi (JP)

(21) Appl. No.: 15/641,444

(22) Filed: Jul. 5, 2017

(30) Foreign Application Priority Data
Jul. 27, 2016 (JP) 2016-147484

(51) Int. Cl. A61K 8/02; A61K 8/37; A61Q 1/00; A61K 8/92

(52) U.S. Cl. CPC A61K 8/022; A61K 8/927; A61K 8/37; A61Q 1/00

(57) ABSTRACT

Provided is a method for producing a solid powder cosmetic material having excellent usability, impact resistance, and water repellency with good moldability in a wet process using water as a dispersion medium. The method includes a step of preparing a slurry by mixing a cosmetic base material containing a powder ingredient and an oily ingredient and a dispersion medium containing water and a step of compression-molding and drying the slurry packed in a container. The oily ingredient contains an ester oil that is a liquid at 25° C. and has a molecular weight of 300 to 600 and an IOB value of 0.5 or less and beeswax. The powder ingredient contains a hydrophobic powder.

### BACKGROUND OF THE INVENTION

### 1. Field of the Invention

The present invention relates to a method for producing a solid powder cosmetic material and a solid powder cosmetic material.

### 2. Description of the Related Art

Solid powder cosmetic materials are widely used in makeup cosmetics, such as eye colors and foundations. A solid powder cosmetic material is generally produced by mixing a powder ingredient containing, for example, an extender pigment, such as mica, a color pigment, or a pearl pigment and an oily ingredient containing, for example, oil or wax and packing and molding this mixture in an inner tray, such as a metal tray or a resin tray. The inner tray containing the molded solid powder cosmetic material is set to, for example, a compact container and is used as a portable cosmetic.

The methods for producing solid powder cosmetic materials are roughly classified into a dry process and a wet process. Recently, excellent usability, such as smoothness, adhesiveness, and spreadability, is required in many cases, leading to a tendency to employ a wet process, which allows a large amount of flaky powder or spherical powder to be blended. Preparation of a slurry in the wet process uses an organic solvent, such as isoparaffin and alcohol, but in consideration of, for example, the environment, workers, and the burden on the skin, use of water as a dispersion medium is increasing.

Incidentally, many of cosmetics, such as foundations, contain hydrophobic powders for, for example, improving the touch, preventing the dullness, and preventing makeup from smudging. However, such a cosmetic base material containing a hydrophobic powder is hardly dispersed in water, which decreases moldability, leading to occurrence of cracking or cleaving in the surface of a molded product or a tendency to decrease the impact resistance of a molded product.

There have been attempts to improve the dispersibility of cosmetic base materials in water. For example, Japanese Unexamined Patent Application Publication No. 2009-242282 discloses a method for preparing a slurry by mixing an oil-in-water type emulsion composition and a cosmetic base material mainly composed of a powder. In addition, Japanese Patent Nos. 4594075 and 5342195 disclose methods for preparing slurries by adding a hydrophilic surfactant or a polyhydric alcohol as an oily ingredient to a cosmetic base material mainly composed of a powder.

### PRIOR ART DOCUMENTS

### Patent Documents

[REFERENCE 1]JP2009-242282
[REFERENCE 2]JP4594075
[REFERENCE 3]JP5342195

### SUMMARY OF THE INVENTION

### Problem to be Solved by the Invention

However, the solid powder cosmetic materials described in the above-mentioned patent documents have problems that the hydrophilic components contained in the cosmetic base materials decrease the water repellency of coated layers, which makes the coated layers vulnerable to water and perspiration.

It is an object of the present invention to provide a method for producing a solid powder cosmetic material having excellent usability, impact resistance, and water repellency with good moldability in a wet process using water as a dispersion medium. It is also an object of the present invention to provide a solid powder cosmetic material having excellent usability, impact resistance, and water repellency and also capable of being molded into a product having excellent appearance.

The present inventors have diligently studied to solve the above problems and have found that a homogeneous slurry can be prepared by mixing a powder ingredient and an oily ingredient containing a specific ester oil and a specific wax to form a cosmetic base material, adding water to the cosmetic base material, and kneading the resulting mixture. The inventors have further found that a solid powder cosmetic material sufficiently suppressed from causing cracking or cleaving can be prepared by packing the slurry in an inner tray and compression-molding and then drying the slurry and that this solid powder cosmetic material shows excellent usability, impact resistance, and water repellency. Thus, the present invention has been accomplished.

That is, the present invention provides a method for producing a solid powder cosmetic material. The method includes a step of preparing a slurry by mixing a cosmetic base material containing a powder ingredient and an oily ingredient and a dispersion medium containing water and a step of compression-molding and drying the slurry packed in a container, wherein the oily ingredient contains an ester oil component (A) that is a liquid at 25° C. and has a molecular weight of 300 to 600 and an inorganic/organic balance (IOB) value of 0.5 or less and a beeswax component (B); and the powder ingredient contains a hydrophobic powder component (C).

From the viewpoint of capable of further improving the usability and the moldability, the content of the component (A) in the cosmetic base material is preferably 5% to 25% by mass based on the total amount of the cosmetic base material.

From the viewpoint of simultaneously achieving higher impact resistance and a better slurry state, the content of the component (B) in the cosmetic base material is preferably 0.3% to 5% by mass based on the total amount of the cosmetic base material.

In the method for producing a solid powder cosmetic material according to the present invention, the slurry may be prepared by mixing the cosmetic base material and the dispersion medium at 60° C. to 95° C.

From the viewpoint of capable of further improving the water repellency, the content of the component (C) in the cosmetic base material is preferably 20% to 94.5% by mass based on the total amount of the cosmetic base material.

The present invention also provides a solid powder cosmetic material containing a powder ingredient and an oily ingredient. The oily ingredient contains an ester oil component (A) that is a liquid at 25° C. and has a molecular weight of 300 to 600 and an IOB value of 0.5 or less and a beeswax component (B). The powder ingredient contains a hydrophobic powder component (C).

From the viewpoint of capable of further improving the usability and the moldability, the content of the component (A) is preferably 5% to 25% by mass based on the total amount of the cosmetic material.

From the viewpoint of simultaneously achieving higher impact resistance and a better slurry state, the content of the component (B) is preferably 0.3% to 5% by mass based on the total amount of the cosmetic material.

From the viewpoint of capable of further improving the water repellency, the content of the component (C) is preferably 20% to 94.5% by mass based on the total amount of the cosmetic material.

The solid powder cosmetic material according to the present invention preferably does not substantially contain a surfactant having an IOB value of 0.7 or more, from the viewpoint of capable of further improving the water repellency.

### Effect of the Invention

An aspect of the present invention can provide a method for producing a solid powder cosmetic material having excellent usability, impact resistance, and water repellency with good moldability in a wet process using water as a dispersion medium.

The solid powder cosmetic material of the present invention has excellent usability, impact resistance, and water repellency and also can be molded into a product having excellent appearance.

### DESCRIPTION OF THE PREFERRED EMBODIMENTS

The solid powder cosmetic material of the present embodiment contains a powder ingredient and an oily ingredient.

The powder ingredient may be any powder that is usually used for cosmetic materials. The powder may have any shape. For example, the powder may have a spherical, plate-like, or acicular shape, may be haze-like, may be fine particles, may have a pigment-grade particle diameter, or may have a porous or nonporous particle structure.

Examples of the powder ingredient include inorganic powders, brilliant powders, organic powders, complex powders, and metal soap powders, specifically, brilliant powders, such as titanium oxide, ferric ferrocyanide, ultramarine blue, red iron oxide, yellow iron oxide, black iron oxide, zinc oxide, aluminum oxide, silica, magnesium oxide, zirconium oxide, magnesium carbonate, calcium carbonate, chromium oxide, aluminum magnesium hydroxide, mica, synthetic mica, sericite, talc, kaolin, silicon carbide, barium sulfate, bentonite, smectite, silicon nitride, bismuth oxychloride, titanium oxide-coated mica, iron oxide-coated mica, iron oxide-coated mica titanium, organic pigment-coated mica titanium, and aluminum powder; organic powders, such as nylon powder, polymethyl methacrylate powder, acrylonitrile-methacrylic acid copolymer powder, vinylidene chloride-methacrylic acid copolymer powder, polyethylene powder, polystyrene powder, organopolysiloxane elastomer powder, polymethylsilsesquioxane powder, polyurethane powder, wool powder, silk powder, crystalline cellulose powder, and N-acyllysine powder; dye powders, such as organic tar-based pigments and lake pigments of organic dyes; complex powders, such as fine particle titanium oxide-coated mica titanium, barium sulfate-coated mica titanium, titanium oxide-containing silica, and zinc oxide-containing silica; and metal soap powders, such as magnesium stearate, zinc myristate, aluminum stearate, and calcium stearate.

The powder ingredients can be used alone or in combination of two or more thereof.

In the solid powder cosmetic material of the embodiment, the powder ingredient preferably contains a hydrophobic powder (may be also referred to as component (C)) from the viewpoint of capable of further improving the water repellency.

The hydrophobic powder may be any powder having a surface exhibiting hydrophobicity, and examples thereof include powders themselves having hydrophobicity and hydrophobized powders, such as hydrophilic powders having surfaces treated with known hydrophobizing agents and powders themselves having hydrophobicity and further treated with hydrophobizing agents for further enhancing the hydrophobicity. Among these powders, examples of the hydrophobic powder itself having hydrophobicity include organic resin powders, such as polystyrene powder, polyethylene powder, N-acyllysine powder, and epoxy resin powder; and metal soap powders, such as aluminum stearate, zinc laurate, and magnesium stearate.

Examples of the hydrophobized powder include powders that are used for cosmetic materials and hydrophobized with materials, such as silicones, fluorine compounds, metal soaps, oils, and acyl glutamic acid, by known methods. These hydrophobized powders can be used alone or in combination of two or more thereof.

The content of the powder ingredient in the solid powder cosmetic material of the embodiment can be 20% to 94.7% by mass based on the total amount of the cosmetic material and is preferably 25% to 93% by mass from the viewpoint of the usability and the water repellency.

The content of the hydrophobic powder in the solid powder cosmetic material of the embodiment can be 20% to 94.5% by mass based on the total amount of the cosmetic material and is preferably 30% to 90% by mass from the viewpoint of the usability.

From the viewpoint of the usability and the water repellency, the content of the hydrophobic powder in the powder ingredient is preferably 21% to 100% by mass, more preferably 30% to 100% by mass, and most preferably 40% to 100% by mass based on the total amount of the powder ingredient.

In the embodiment, the oily ingredient preferably contains an ester oil component (A) (may be also referred to as component (A)) that is a liquid at 25° C. and has a molecular weight of 300 to 600 and an IOB value of 0.5 or less and a beeswax component (B) (may be also referred to as component (B)). A combination of these components can provide a homogeneous slurry state when a solid powder cosmetic material is produced by a wet process using a dispersion containing water and can realize a solid powder cosmetic material having improved moldability and excellent usability and impact resistance.

Examples of the component (A) include octyl palmitate (MW: 368, IOB: 0.13), glyceryl tri(2-ethylhexanoate) (MW: 471, IOB: 0.35), octyl stearate (MW: 390, IOB: 0.12), cetyl ethylhexanoate (MW: 369, IOB: 0.13), and neopentyl glycol di(2-ethylhexanoate) (MW: 357, IOB: 0.47).

In the specification, the IOB value is a well-known value representing the ratio of an inorganic value to an organic value determined based on an organic conceptual diagram and represents the degree of polarity of an oily base and is represented by the following expression (I):

IOB=inorganic value (IV)/organic value (OV) (I).

Specifically, the IOB can be determined by the expression (I) in accordance with the description in “Yuki kagobutsu no yosoku to yuki gainen zu (Estimation of organic compound and organic conceptual diagram)”, Fujita, Kagaku no Ryoiki, Vol. 11, No. 10, 1957, pp. 719 to 725; and “Yuki gainen zu niyoru nyuka syoho sekkei (Emulsification formulation design by organic conceptual diagram)”, Nihon Emulsion Co., Ltd., Yaguchi, 1985, p. 98.

The component (A) preferably has a molecular weight of 300 to 500 from the viewpoint of the usability and the moldability.

The ester oils as the component (A) may be used alone or in combination of two or more thereof.

The content of the component (A) is preferably 5% to 25% by mass based on the total amount of the cosmetic material from the viewpoint of capable of further improving the usability and the moldability and is more preferably 6% to 20% by mass from the viewpoint of the usability and the impact resistance.

The component (B) may be any beeswax that is used for cosmetic materials.

The content of the component (B) is preferably 0.3% to 5.0% by mass based on the total amount of the cosmetic material from the viewpoint of simultaneously achieving higher impact resistance and a better slurry state and is more preferably 1.0% to 4.0% by mass from the viewpoint of the homogeneity of the slurry in a wet process and the usability.

As an oily ingredient other than the components (A) and (B), any oily ingredient that is usually used for cosmetic materials can be used. For example, oils and fats, hydrogenated oils, fatty acids, higher alcohols, silicone oils, fluorine oils, lanolin derivatives, and oil gelling agents can be used regardless of the origins, such as animal oil, plant oil, and synthetic oil, and the properties, such as solid oil, semisolid oil, liquid oil, and volatile oil.

The solid powder cosmetic material of the embodiment can further contain any material that is usually used for a cosmetic material, such as an antiseptic, an antioxidant, a dye, a thickener, a pH adjuster, a perfume, an UV absorber, and a humectant, in addition to the above-mentioned components.

The solid powder cosmetic material of the embodiment preferably does not substantially contain a surfactant having an IOB value of 0.7 or more, from the viewpoint of capable of further improving the water repellency. Examples of the surfactant herein include anionic surfactants, such as fatty acid soap, higher alkyl sulfate ester salts, and alkyl ether sulfate ester salts; cationic surfactants, such as alkyl trimethyl ammonium salts and alkyl pyridinium salts; amphoteric surfactants, such as alkyldimethylaminoacetic acid betaine; nonionic surfactants, such as sorbitan fatty acid esters and POE-sorbitan fatty acid esters; and polyhydric alcohols, such as glycerin, 1,3-butylene glycol, dipropylene glycol, and polyalkylene glycol. The term “not substantially containing” refers to a range giving a sufficient water repellency of the solid powder cosmetic material. From such a viewpoint, the content of the surfactant having an IOB value of 0.7 or more is preferably 0.5% by mass or less and more preferably 0% by mass.

When water is dropped on a coating film of the solid powder cosmetic material of the embodiment formed on a base material under an environment of 25° C., the contact angle of water at 1 minute after the dropping is preferably 100° or more, more preferably 110° or more. Herein, the term “contact angle” refers to the angle formed between a water droplet and a coating film and refers to the numerical value obtained by a tangent method (Tangent 1).

The solid powder cosmetic material of the embodiment is suitable for, for example, a makeup cosmetic, such as a foundation, a face color, an eye shadow, and a blusher.

A method for producing a solid powder cosmetic material of the present embodiment will now be described.

The method for producing a solid powder cosmetic material of the embodiment includes a step of preparing a slurry by mixing a cosmetic base material containing a powder ingredient and an oily ingredient and a dispersion medium containing water and a step of compression-molding and drying the slurry packed in a container.

Examples of the powder ingredient and the oily ingredient are the same as those mentioned above, and their amounts in the cosmetic base material can be the same as the preferred ranges in the above-described solid powder cosmetic material. The composition of the cosmetic base material excluding the powder ingredient and the oily ingredient can also be the same as the preferred composition of the above-described solid powder cosmetic material.

The cosmetic base material according to the embodiment can be prepared by, for example, a method including a step of preparing a first mixture containing a powder ingredient, a step of preparing a second mixture containing an oily ingredient, and a step of mixing the first mixture and the second mixture.

The step of preparing a first mixture can be carried out with, for example, a super mixer or Henschel mixer and may optionally include pulverization with an atomizer.

The step of preparing a second mixture can be carried out with, for example, a disperser or homomixer and can mix an oily ingredient while heating at 60° C. to 80° C., preferably at 60° C. to 70° C.

The step of mixing the first mixture and the second mixture can be carried out with, for example, a super mixer or Henschel mixer and may optionally include pulverization with an atomizer.

Examples of the method for preparing a slurry include a method involving adding a dispersion medium containing a predetermined amount of water to the cosmetic base material prepared above and mixing them.

The blending ratio between the cosmetic base material and the dispersion medium, cosmetic base material: dispersion medium as the mass ratio, can be 100:80 to 100:150 and is preferably 100:100 to 100:140 from the viewpoint of the moldability.

The content of water in the dispersion medium can be 90% to 100% by mass and is preferably 95% to 100% by mass from the viewpoint of the water repellency.

As the dispersion medium other than water, an aqueous solvent, such as ethyl alcohol, acetone, and isopropyl alcohol, can be used, but since such an aqueous solvent tends to slightly decrease the water repellency, water alone is preferred.

Examples of the method for mixing the cosmetic base material and the dispersion medium include a method involving kneading with, for example, a kneader or versatile stirrer. The mixing can be carried out with heating at 60° C. to 95° C., preferably at 60° C. to 80° C.

In the step of compression-molding and drying the slurry packed in a container, the slurry prepared above can be optionally degassed and can be packed in a predetermined container and then be compression-molded and dried.

The predetermined container is, for example, an inner tray, and examples of the inner tray include metal trays and resin trays.

Degassing of the slurry can be performed, for example, under conditions of a pressure of 0.06 to 0.10 mPa for 5 to 10 minutes.

The compression-molding can be performed, for example, under conditions of 3 to 6 kgf/cm² and room temperature. The drying can be performed, for example, in a dryer at 40° C. to 70° C. for 5 to 12 hours.

In the compression-molding and the drying, the compression-molding is carried out first, and then the drying may be carried out; the drying is carried out first, and then the compression-molding may be carried out; the compression-molding may be carried out with drying; or these procedures may be combined.

The solid powder cosmetic material having a predetermined shape according to the embodiment can be prepared through the above-described steps.

### EXAMPLES

The present invention will now be described in more detail by examples, but the technical scope of the invention is not limited to the following examples. Incidentally, the numerical values in the tables indicate the contents (% by mass) based on the total amount of the cosmetic base material (total amount of the components excluding water). The numerical values relating to water indicate the proportions (parts by mass) with respect to 100 parts by mass of the total amount of the cosmetic base material (total amount of the components excluding water).

Prior to the explanation of the examples, the evaluation method employed in each example will be described.

### (1) Usability

Twenty expert panelists for evaluation of cosmetics used the foundations (solid powder cosmetics) of Examples and Comparative Examples and evaluated the “smoothness”, “adhesiveness”, and “spreadability” of each sample in accordance with the following evaluation criteria and scored from one to five. The average of the scores given by all the panelists was judged by the following criteria.

### [Score: Evaluation Criteria]

5: Excellent
4: Good
3: Average
2: Fair
1: Poor

### [Judgment Criteria (Average of Scores)]

Excellent: 4.5 or more
Good: 3.5 or more and less than 4.5
Fair: 1.5 or more and less than 3.5
Poor: less than 1.5

### (2) Impact Resistance

Each cosmetic compact container packed with the solid powder cosmetic material was dropped from a height of 50 cm onto tile 5 times, and the surface state was observed to evaluate by the following evaluation criteria.

### [Evaluation Criteria]

Excellent: No change is observed,
Good: Slight cracking or peeling is observed,
Fair: Cracking or peeling is observed, and
Poor: Significant cracking or peeling is observed.

### (3) Slurry State

A slurry (1.0 g) prepared by adding water to a cosmetic base material and mixing it was applied and spread onto a slide glass, and the surface state was visually observed and was judged by the following four judgment criteria.

### [Judgment Criteria]

Excellent: There are no lumps of powder, and the powder is finely mixed with water,
Good: There are no lumps of powder, but the powder is roughly mixed with water,
Fair: Lumps of powder slightly remain, and
Poor: Lumps of powder remain.

### (4) Moldability

A slurry was packed in an inner tray and was then compression-molded and dried. The resulting molded product was visually observed for cracking, peeling, cleaving, and other defects occurring on the surface, and the level of occurrence was judged by the following four judgment criteria.

### [Judgment Criteria]

Excellent: No occurrence is observed,
Good: Occurrence is slightly observed,
Fair: Occurrence is observed, and
Poor: The occurrence rate is very high.

### (5) Water Repellency

A slurry was packed in an inner tray and was then compression-molded and dried, and 0.5 g of the resulting molded product was scraped off and was applied onto artificial skin at a uniform thickness with an exclusive chip. Under an environment of 25° C., water was dropped onto this coating film, and the contact angle was measured after one minute with a contact angle measuring device DSA30 (manufactured by KRUSS GmbH). Herein, the term “contact angle” refers to the angle formed between a water droplet and a coating film and refers to the numerical value obtained by a tangent method (Tangent 1).

### [Evaluation Criteria]

Excellent: a contact angle of 110° or more,
Good: a contact angle of 100° or more and less than 110°,
Fair: a contact angle of 90° or more and less than 100°, and
Poor: a contact angle of less than 90°.

### Examples 1 to 15 and Comparative Examples 2, 3, and 5 to 10

Foundations having the compositions shown in Tables 1 to 5 were prepared by the following process and were evaluated for usability, impact resistance, slurry state, moldability, and water repellency. The results are also shown in Tables 1 to 5.

### Process for Preparation

Components 1 to 14 were uniformly dispersed with a Henschel mixer to obtain a mixture I. Separately, components 15 to 27 were mixed and were heated to 60° C. to 70° C. (80° C. to 86° C. in Comparative Example 2 and 68° C. to 72° C. in Comparative Example 3) to obtain a mixture II. Subsequently, the mixture II was added to the mixture I, and the resulting mixture was uniformly dispersed with a Henschel mixer to obtain a cosmetic base material. Water was added to this cosmetic base material, followed by heating to 60° C. and kneading with a kneader. The resulting slurry was degassed at 0.08 mPa for 5 minutes, was then packed in an inner tray (metal tray), was compression-molded at 5 kgf/cm², and was then dried in a dryer at 40° C. for 10 hours to obtain a foundation sample.

### Comparative Example 1

A foundation having the composition shown in Table 4 was tried to be prepared by the following process, but the slurry could not be produced.

Components 1 to 14 were uniformly dispersed with a Henschel mixer to obtain a mixture I. Separately, components 15 to 27 were mixed at 60° C. to 70° C. to obtain a mixture II. Subsequently, the mixture II was added to the mixture I, and the resulting mixture was dispersed with a Henschel mixer to obtain a cosmetic base material. Water was added to this cosmetic base material, followed by heating to 60° C. and kneading with a kneader. However, no slurry was produced.

### Comparative Example 4

A foundation having the composition shown in Table 4 was tried to be prepared by the following process, but the slurry could not be produced.

Components 1 to 14 were uniformly dispersed with a Henschel mixer to obtain a mixture I. Separately, components 15 to 27 were mixed at 60° C. to 70° C. to obtain a mixture II. Subsequently, the mixture II was added to the mixture I, and the resulting mixture was dispersed with a Henschel mixer to obtain a cosmetic base material. Water was added to this cosmetic base material, followed by kneading at room temperature (25° C.) with a kneader. However, no slurry was produced.

### Comparative Examples 11 to 14

Foundations having the compositions shown in Table 6 were prepared by the following process and were evaluated for usability, impact resistance, slurry state, moldability, and water repellency. The results are also shown in Table 6.

### Process for Preparation

Components 1 to 8 were uniformly dispersed with a Henschel mixer to obtain a mixture I. Separately, components 9 to 13 were mixed and were heated to 60° C. to 70° C. to obtain a mixture II. Subsequently, the mixture II was added to the mixture I, and the resulting mixture was uniformly dispersed with a Henschel mixer to obtain a cosmetic base material. Water was added to this cosmetic base material, followed by heating to 60° C. and kneading with a kneader. The resulting slurry was degassed at 0.08 mPa for 5 minutes, was then packed in an inner tray (metal tray), was compression-molded at 5 kgf/cm², and was then dried in a dryer at 40° C. for 10 hours to obtain a foundation sample.

TABLE 1

| | | Example | | | | |
|---|---|---|---|---|---|---|
| No. | Component | 1 | 2 | 3 | 4 | 5 |
| 1 | Silicone-treated synthetic mica | 45.00 | 45.00 | 45.00 | 45.00 | 45.00 |
| 2 | Untreated talc | — | — | — | — | — |
| 3 | Silicone-treated talc | 14.90 | 16.90 | 14.90 | 6.90 | 14.90 |
| 4 | Fluorine-treated boron nitride | 10.00 | 10.00 | 10.00 | 10.00 | 10.00 |
| 5 | Boron nitride | — | — | — | — | — |
| 6 | Repellent ultrafine titanium oxide | 10.00 | 10.00 | 10.00 | 10.00 | 10.00 |
| | particle-nylon complex powder | | | | | |
| 7 | Silicone-treated yellow iron oxide | 3.00 | 3.00 | 3.00 | 3.00 | 3.00 |
| 8 | Silicone-treated red iron oxide | 1.00 | 1.00 | 1.00 | 1.00 | 1.00 |
| 9 | Silicone-treated black iron oxide | 0.10 | 0.10 | 0.10 | 0.10 | 0.10 |
| 10 | Silicone-treated titanium oxide | 8.00 | 8.00 | 8.00 | 8.00 | 8.00 |
| 11 | Untreated yellow iron oxide | — | — | — | — | — |
| 12 | Untreated red iron oxide | — | — | — | — | — |
| 13 | Untreated black iron oxide | — | — | — | — | — |
| 14 | Untreated titanium oxide | — | — | — | — | — |
| 15 | Octyl stearate | 7.50 | 5.00 | 7.00 | 15.00 | 7.50 |
| 16 | Octyl palmitate | — | — | — | — | — |
| 17 | Glyceryl tri(2-ethylhexanoate) | — | — | — | — | — |
| 18 | Neopentyl glycol di-2-ethylhexanoate | — | — | — | — | — |
| 19 | Diisostearyl malate | — | — | — | — | — |
| 20 | PEG-30 glyceryl isostearate | — | — | — | — | — |
| 21 | PEG-20 glyceryl triisostearate | — | — | — | — | — |
| 22 | PEG-20 sorbitan isostearate | — | — | — | — | — |
| 23 | PEG-40 hydrogenated castor | — | — | — | — | — |
| 24 | Dimethicone | — | — | — | — | — |
| 25 | Beeswax | 0.50 | 1.00 | 1.00 | 1.00 | 1.50 |
| 26 | Carnauba wax | — | — | — | — | — |
| 27 | Candelilla wax | — | — | — | — | — |
| 28 | Water (parts by mass) | 130 | 130 | 130 | 110 | 130 |

| | | | | | |
|---|---|---|---|---|---|
| Feelings in use | Excellent | Excellent | Excellent | Excellent | Excellent |
| Impact resistance | Excellent | Excellent | Excellent | Excellent | Excellent |
| Slurry state | Good | Good | Excellent | Excellent | Excellent |
| Moldability | Excellent | Excellent | Excellent | Excellent | Excellent |
| Water repellency | Excellent | Excellent | Excellent | Excellent | Excellent |

TABLE 2

| | | Example | | | | |
|---|---|---|---|---|---|---|
| No. | Component | 6 | 7 | 8 | 9 | 10 |
| 1 | Silicone-treated synthetic mica | 45.00 | 45.00 | 45.00 | 45.00 | 20.00 |
| 2 | Untreated talc | — | — | — | — | 39.90 |
| 3 | Silicone-treated talc | 14.90 | 14.90 | 14.90 | 14.90 | — |
| 4 | Fluorine-treated boron nitride | 10.00 | 10.00 | 10.00 | 10.00 | — |
| 5 | Boron nitride | — | — | — | — | 10.00 |
| 6 | Repellent ultrafine titanium oxide | 10.00 | 10.00 | 10.00 | 10.00 | 10.00 |
| | particle-nylon complex powder | | | | | |
| 7 | Silicone-treated yellow iron oxide | 3.00 | 3.00 | 3.00 | 3.00 | — |
| 8 | Silicone-treated red iron oxide | 1.00 | 1.00 | 1.00 | 1.00 | — |
| 9 | Silicone-treated black iron oxide | 0.10 | 0.10 | 0.10 | 0.10 | — |
| 10 | Silicone-treated titanium oxide | 8.00 | 8.00 | 8.00 | 8.00 | — |
| 11 | Untreated yellow iron oxide | — | — | — | — | 3.00 |
| 12 | Untreated red iron oxide | — | — | — | — | 1.00 |
| 13 | Untreated black iron oxide | — | — | — | — | 0.10 |
| 14 | Untreated titanium oxide | — | — | — | — | 8.00 |
| 15 | Octyl stearate | 10.00 | 15.00 | 20.00 | 25.00 | 7.00 |
| 16 | Octyl palmitate | — | — | — | — | — |
| 17 | Glyceryl tri(2-ethylhexanoate) | — | — | — | — | — |
| 18 | Neopentyl glycol di-2-ethylhexanoate | — | — | — | — | — |
| 19 | Diisostearyl malate | — | — | — | — | — |
| 20 | PEG-30 glyceryl isostearate | — | — | — | — | — |
| 21 | PEG-20 glyceryl triisostearate | — | — | — | — | — |
| 22 | PEG-20 sorbitan isostearate | — | — | — | — | — |
| 23 | PEG-40 hydrogenated castor | — | — | — | — | — |
| 24 | Dimethicone | — | — | — | — | — |
| 25 | Beeswax | 2.00 | 3.00 | 4.00 | 5.00 | 1.00 |
| 26 | Carnauba wax | — | — | — | — | — |
| 27 | Candelilla wax | — | — | — | — | — |
| 28 | Water (parts by mass) | 130 | 110 | 110 | 100 | 130 |

| | | | | | |
|---|---|---|---|---|---|
| Feelings in use | Excellent | Excellent | Good | Good | Excellent |
| Impact resistance | Excellent | Excellent | Excellent | Good | Excellent |
| Slurry state | Excellent | Excellent | Excellent | Excellent | Excellent |
| Moldability | Excellent | Excellent | Excellent | Good | Good |
| Water repellency | Excellent | Excellent | Good | Good | Good |

TABLE 3

| | | Example | | | | |
|---|---|---|---|---|---|---|
| No. | Component | 11 | 12 | 13 | 14 | 15 |
| 1 | Silicone-treated synthetic mica | 30.00 | 45.00 | 45.00 | 45.00 | 45.00 |
| 2 | Untreated talc | 29.90 | — | — | — | — |
| 3 | Silicone-treated talc | — | 14.90 | 14.90 | 14.90 | 14.90 |
| 4 | Fluorine-treated boron nitride | — | 10.00 | 10.00 | 10.00 | 10.00 |
| 5 | Boron nitride | 10.00 | — | — | — | — |
| 6 | Repellent ultrafine titanium oxide | 10.00 | 10.00 | 10.00 | 10.00 | 10.00 |
| | particle-nylon complex powder | | | | | |
| 7 | Silicone-treated yellow iron oxide | — | 3.00 | 3.00 | 3.00 | 3.00 |
| 8 | Silicone-treated red iron oxide | — | 1.00 | 1.00 | 1.00 | 1.00 |
| 9 | Silicone-treated black iron oxide | — | 0.10 | 0.10 | 0.10 | 0.10 |
| 10 | Silicone-treated titanium oxide | — | 8.00 | 8.00 | 8.00 | 8.00 |
| 11 | Untreated yellow iron oxide | 3.00 | — | — | — | — |
| 12 | Untreated red iron oxide | 1.00 | — | — | — | — |
| 13 | Untreated black iron oxide | 0.10 | — | — | — | — |
| 14 | Untreated titanium oxide | 8.00 | — | — | — | — |
| 15 | Octyl stearate | 7.00 | 10.00 | — | — | — |
| 16 | Octyl palmitate | — | — | 10.00 | — | — |
| 17 | Glyceryl tri(2-ethylhexanoate) | — | — | — | 10.00 | — |
| 18 | Neopentyl glycol di-2-ethylhexanoate | — | — | — | — | 10.00 |
| 19 | Diisostearyl malate | — | — | — | — | — |
| 20 | PEG-30 glyceryl isostearate | — | — | — | — | — |
| 21 | PEG-20 glyceryl triisostearate | — | — | — | — | — |
| 22 | PEG-20 sorbitan isostearate | — | — | — | — | — |
| 23 | PEG-40 hydrogenated castor | — | — | — | — | — |
| 24 | Dimethicone | — | — | — | — | — |
| 25 | Beeswax | 1.00 | 2.00 | 2.00 | 2.00 | 2.00 |
| 26 | Carnauba wax | — | — | — | — | — |
| 27 | Candelilla wax | — | — | — | — | — |
| 28 | Water (parts by mass) | 130 | 130 | 130 | 130 | 130 |

| | | | | | |
|---|---|---|---|---|---|
| Feelings in use | Excellent | Excellent | Excellent | Excellent | Excellent |
| Impact resistance | Excellent | Excellent | Excellent | Excellent | Excellent |
| Slurry state | Excellent | Excellent | Excellent | Excellent | Excellent |
| Moldability | Good | Excellent | Excellent | Excellent | Excellent |
| Water repellency | Excellent | Excellent | Excellent | Excellent | Excellent |

TABLE 4

| | | Comparative Example | | | | |
|---|---|---|---|---|---|---|
| No. | Component | 1 | 2 | 3 | 4 | 5 |
| 1 | Silicone-treated synthetic mica | 45.00 | 45.00 | 45.00 | 45.00 | 45.00 |
| 2 | Untreated talc | — | — | — | — | — |
| 3 | Silicone-treated talc | 14.90 | 14.90 | 14.90 | 14.90 | 14.90 |
| 4 | Fluorine-treated boron nitride | 10.00 | 10.00 | 10.00 | 10.00 | 10.00 |
| 5 | Boron nitride | — | — | — | — | — |
| 6 | Repellent ultrafine titanium oxide | 10.00 | 10.00 | 10.00 | 10.00 | 10.00 |
| | particle-nylon complex powder | | | | | |
| 7 | Silicone-treated yellow iron oxide | 3.00 | 3.00 | 3.00 | 3.00 | 3.00 |
| 8 | Silicone-treated red iron oxide | 1.00 | 1.00 | 1.00 | 1.00 | 1.00 |
| 9 | Silicone-treated black iron oxide | 0.10 | 0.10 | 0.10 | 0.10 | 0.10 |
| 10 | Silicone-treated titanium oxide | 8.00 | 8.00 | 8.00 | 8.00 | 8.00 |
| 11 | Untreated yellow iron oxide | — | — | — | — | — |
| 12 | Untreated red iron oxide | — | — | — | — | — |
| 13 | Untreated black iron oxide | — | — | — | — | — |
| 14 | Untreated titanium oxide | — | — | — | — | — |
| 15 | Octyl stearate | 8.00 | 7.00 | 7.00 | 7.00 | — |
| 16 | Octyl palmitate | — | — | — | — | — |
| 17 | Glyceryl tri(2-ethylhexanoate) | — | — | — | — | — |
| 18 | Neopentyl glycol di-2-ethylhexanoate | — | — | — | — | — |
| 19 | Diisostearyl malate | — | — | — | — | 10.00 |
| 20 | PEG-30 glyceryl isostearate | — | — | — | — | — |
| 21 | PEG-20 glyceryl triisostearate | — | — | — | — | — |
| 22 | PEG-20 sorbitan isostearate | — | — | — | — | — |
| 23 | PEG-40 hydrogenated castor | — | — | — | — | — |
| 24 | Dimethicone | — | — | — | — | — |
| 25 | Beeswax | — | — | — | 1.00 | — |
| 26 | Carnauba wax | — | 1.00 | — | — | — |
| 27 | Candelilla wax | — | — | 1.00 | — | — |
| 28 | Water (parts by mass) | 130 | 130 | 130 | 130 | 130 |

| | | | | | |
|---|---|---|---|---|---|
| Feelings in use | — | Fair | Fair | — | Fair |
| Impact resistance | — | Fair | Fair | — | Excellent |
| Slurry state | Poor | Good | Good | Poor | Fair |
| Moldability | Poor | Fair | Fair | Poor | Fair |
| Water repellency | — | Good | Excellent | — | Excellent |

TABLE 5

| | | Comparative Example | | | | |
|---|---|---|---|---|---|---|
| No. | Component | 6 | 7 | 8 | 9 | 10 |
| 1 | Silicone-treated synthetic mica | 45.00 | 45.00 | 45.00 | 45.00 | 45.00 |
| 2 | Untreated talc | — | — | — | — | — |
| 3 | Silicone-treated talc | 14.90 | 14.90 | 14.90 | 14.90 | 14.90 |
| 4 | Fluorine-treated boron nitride | 10.00 | 10.00 | 10.00 | 10.00 | 10.00 |
| 5 | Boron nitride | — | — | — | — | — |
| 6 | Repellent ultrafine titanium oxide | 10.00 | 10.00 | 10.00 | 10.00 | 10.00 |
| | particle-nylon complex powder | | | | | |
| 7 | Silicone-treated yellow iron oxide | 3.00 | 3.00 | 3.00 | 3.00 | 3.00 |
| 8 | Silicone-treated red iron oxide | 1.00 | 1.00 | 1.00 | 1.00 | 1.00 |
| 9 | Silicone-treated black iron oxide | 0.10 | 0.10 | 0.10 | 0.10 | 0.10 |
| 10 | Silicone-treated titanium oxide | 8.00 | 8.00 | 8.00 | 8.00 | 8.00 |
| 11 | Untreated yellow iron oxide | — | — | — | — | — |
| 12 | Untreated red iron oxide | — | — | — | — | — |
| 13 | Untreated black iron oxide | — | — | — | — | — |
| 14 | Untreated titanium oxide | — | — | — | — | — |
| 15 | Octyl stearate | — | — | — | — | — |
| 16 | Octyl palmitate | — | — | — | — | — |
| 17 | Glyceryl tri(2-ethylhexanoate) | — | — | — | — | — |
| 18 | Neopentyl glycol di-2-ethylhexanoate | — | — | — | — | — |
| 19 | Diisostearyl malate | — | — | — | — | — |
| 20 | PEG-30 glyceryl isostearate | 10.00 | — | — | — | — |
| 21 | PEG-20 glyceryl triisostearate | — | 10.00 | — | — | — |
| 22 | PEG-20 sorbitan isostearate | — | — | 10.00 | — | — |
| 23 | PEG-40 hydrogenated castor | — | — | — | 10.00 | — |
| 24 | Dimethicone | — | — | — | — | 7.5 |
| 25 | Beeswax | — | — | — | — | — |
| 26 | Carnauba wax | — | — | — | — | — |
| 27 | Candelilla wax | — | — | — | — | — |
| 28 | Water (parts by mass) | 130 | 130 | 130 | 130 | 140 |

| | | | | | |
|---|---|---|---|---|---|
| Feelings in use | Good | Good | Good | Good | Good |
| Impact resistance | Good | Good | Good | Good | Good |
| Slurry state | Excellent | Excellent | Excellent | Excellent | Fair |
| Moldability | Excellent | Excellent | Excellent | Excellent | Fair |
| Water repellency | Poor | Poor | Poor | Poor | Excellent |

TABLE 6

| | | Comparative Example | | | |
|---|---|---|---|---|---|
| No. | Component | 11 | 12 | 13 | 14 |
| 1 | Silicone-treated synthetic mica | 45.00 | 45.00 | 45.00 | 45.00 |
| 2 | Silicone-treated talc | 14.90 | 14.90 | 14.90 | 14.90 |
| 3 | Fluorine-treated boron nitride | 10.00 | 10.00 | 10.00 | 10.00 |
| 4 | Repellent ultrafine titanium oxide particle- | 10.00 | 10.00 | 10.00 | 10.00 |
| | nylon complex powder | | | | |
| 5 | Silicone-treated yellow iron oxide | 3.00 | 3.00 | 3.00 | 3.00 |
| 6 | Silicone-treated red iron oxide | 1.00 | 1.00 | 1.00 | 1.00 |
| 7 | Silicone-treated black iron oxide | 0.10 | 0.10 | 0.10 | 0.10 |
| 8 | Silicone-treated titanium oxide | 8.00 | 8.00 | 8.00 | 8.00 |
| 9 | Ethylhexyl stearate | 7.00 | 7.00 | 7.00 | 7.00 |
| 10 | Glycerin mono-2-ethylhexyl ether/glycerin | 1.00 | — | — | — |
| | fatty acid ester mixture | | | | |
| 11 | Lipophilic glyceryl monostearate | — | 1.00 | | |
| 12 | PEG-4 distearate | — | — | 1.00 | |
| 13 | PG stearate (SE) | — | — | — | 1.00 |
| 14 | Water (parts by mass) | 130 | 130 | 130 | 130 |

| | | | | |
|---|---|---|---|---|
| Feelings in use | Excellent | Good | Good | Good |
| Impact resistance | Excellent | Poor | Poor | Poor |
| Slurry state | Excellent | Excellent | Excellent | Excellent |
| Moldability | Excellent | Poor | Poor | Poor |
| Water repellency | Poor | Poor | Poor | Poor |

The details of each component shown in Tables 1 to 4 are as follows:

Silicone-treated synthetic mica: PDM-5L(S) (manufactured by Topy Industries, Limited),

Silicone-treated talc: SA Talc JA-13R (manufactured by Miyoshi Kasei Industry Co., Ltd.),

Fluorine-treated boron nitride: FHS-5A BN-SF6 (manufactured by Daito Kasei Kogyo Co., Ltd.),

Boron nitride: RonaFlair Boroneige SF-6 (manufactured by Merck KGaA),

Repellent ultrafine titanium oxide particle-nylon complex powder: FTO60-NL (manufactured by Hayate Material, Y.K.),

Silicone-treated yellow iron oxide: SA-Yellow LL-100P (manufactured by Miyoshi Kasei Industry Co., Ltd.),

Silicone-treated red iron oxide: SA-Bengala No. 216P (manufactured by Miyoshi Kasei Industry Co., Ltd.),

Silicone-treated black iron oxide: SA-Black BL-100P (manufactured by Miyoshi Kasei Industry Co., Ltd.),

Silicone-treated titanium oxide: SA-HOMBITAN AFDC200 (manufactured by Miyoshi Kasei Industry Co., Ltd.),

Untreated yellow iron oxide: TAROX Synthetic Iron Oxide LL-100P (manufactured by Titan Kogyo, Ltd.),

Untreated red iron oxide: Bengala No. 216P (manufactured by Daito Kasei K.K.),

Untreated black iron oxide: TAROX Synthetic Iron Oxide BL-100P (manufactured by Titan Kogyo, Ltd.),

Untreated titanium oxide: HOMBITAN AFDC200 (manufactured by Sachtleben Chemie GmbH),

Octyl stearate: CETIOL 868 (manufactured by BASF Japan Ltd., IOB: 0.12, MW: 390),

Octyl palmitate (IOB: 0.13, MW: 368),

Glyceryl tri(2-ethylhexanoate) (IOB: 0.35, MW: 471),

Neopentyl glycol di(2-ethylhexanoate) (IOB: 0.47, MW: 357),

Diisostearyl malate (IOB: 0.28, MW: 639),

PEG-30 glyceryl isostearate: Emalex GWIS-130 (manufactured by Nihon Emulsion Co., Ltd.) (IOB: 1.46, MW: 1680),

PEG-20 glyceryl triisostearate: Emalex GWIS-320 (manufactured by Nihon Emulsion Co., Ltd.) (IOB: 0.79, MW: 1773),

PEG-20 sorbitan isostearate: NIKKOL TI-10V (manufactured by Nikko Chemicals Co., Ltd.) (IOB: 1.36, MW: 1312),

PEG-40 hydrogenated castor: NIKKOL HCO-40 (manufactured by Nikko Chemicals Co., Ltd.) (IOB: 1.06, MW: 2702),

Dimethicone: KF-96A-100CS (manufactured by Shin-Etsu Chemical Co., Ltd.) (IOB: 0.39, MW: 6600),

Beeswax: Deodorized and Purified High-acid Value Beeswax (manufactured by Cerarica Noda Co., Ltd.),

Carnauba wax: Purified Carnauba Wax R-100 (manufactured by Yokozeki Oil & Fat Industries Co., Ltd.),

Candelilla wax: Purified Candelilla Wax Refine (manufactured by Mitsuba Trading Co., Ltd.),

Glycerin mono-2-ethylhexyl ether/glycerin fatty acid ester mixture: NIKKOL Nikoguard 88 (manufactured by Nikko Chemicals Co., Ltd.) (IOB: 1.18, MW: 218),

Lipophilic glyceryl monostearate: NIKKOL MGS-BV (manufactured by Nikko Chemicals Co., Ltd.) (IOB: 0.63, MW: 359),

PEG-4 distearate: Emalex 200di-S (manufactured by Nihon Emulsion Co., Ltd.) (IOB: 0.39, MW: 727), and

PG stearate (SE): NIKKOL PMS-SE (manufactured by Nikko Chemicals Co., Ltd.) (IOB: 0.4, MW: 388).

As shown in Tables 1 and 2, the solid powder cosmetic materials prepared in Examples 1 to 15 were evaluated to be “Excellent” or “Good” in all of “usability”, “impact resistance”, “slurry state”, “moldability”, and “water repellency”.

### Example 16: Foundation

Component: Blending ratio (% by mass)
1. Silicone-treated synthetic mica: 45.00
2. Silicone-treated talc: 10.90
3. Fluorine-treated boron nitride: 10.00
4. Repellent ultrafine titanium oxide particle-nylon complex powder: 10.00
5. Silicone-treated yellow iron oxide: 3.00
6. Silicone-treated red iron oxide: 1.00
7. Silicone-treated black iron oxide: 0.10
8. Silicone-treated titanium oxide: 8.00
9. Octyl stearate: 10.00
10. Beeswax: 2.00
Water: 130 parts by mass based on 100 parts by mass of the total of the components 1 to 10.

The details of these components are the same as those described above.

### Process for Preparation

Components 1 to 8 were uniformly dispersed with a Henschel mixer and were then pulverized with an atomizer to obtain a mixture I. Separately, components 9 and 10 were mixed and were heated at 60° C. to 70° C. to obtain a mixture II. Subsequently, the mixture II was added to the mixture I, and the resulting mixture was uniformly dispersed with a Henschel mixer and was then pulverized with an atomizer to obtain a cosmetic base material. Water was added to this cosmetic base material, followed by heating to 60° C. and kneading with a kneader. The resulting slurry was degassed at 0.08 mPa for 5 minutes, was then packed in an inner tray (metal tray), was compression-molded at 5 kgf/cm², and was then dried in a dryer at 40° C. for 10 hours to obtain a foundation sample.

### Evaluation

The resulting foundation samples were evaluated as above, and it was demonstrated that the samples were evaluated to be “Excellent” in all of “usability”, “impact resistance”, “slurry state”, “moldability”, and “water repellency”.

### Example 17: Face Color

Component: Blending ratio (% by mass)
1. Silicone-treated synthetic mica: 30.00
2. Calcium carbonate: 10.00
3. Silicic acid anhydride: 15.00
4. Silicone-treated talc: 10.00
5. Sericite: 16.30
6. Silicone-treated yellow iron oxide: 0.30
7. Silicone-treated red iron oxide: 0.30
8. Silicone-treated ultramarine blue: 0.10
9. Titanated mica A: 6.00
10. Titanated mica B: 3.00
11. Octyl stearate: 7.50
12. Beeswax: 1.50
Water: 130 parts by mass based on 100 parts by mass of the total of the components 1 to 12.

The details of these components are the same as those described above, excepting the following components:

Calcium carbonate: Light calcium carbonate (manufactured by Takehara Kagaku Kogyo Co., Ltd.),

Silicic acid anhydride: SUNSIL-130L (manufactured by Sunjin Chemical Co., Ltd.),

Sericite: Eightpearl 300S (manufactured by Kakuhachi Co., Ltd.),

Silicone-treated ultramarine blue: SA-Cosmetic Ultramarine Blue CB-80 (100%) (manufactured by Miyoshi Kasei Industry Co., Ltd.),

Titanated mica A: Timiron MP-115 (manufactured by Merck KGaA), and

Titanated mica B: Timiron MP-1005 (manufactured by Merck KGaA).

### Process for Preparation

Components 1 to 10 were uniformly dispersed with a Henschel mixer to obtain a mixture I. Separately, components 11 and 12 were mixed and were heated at 60° C. to 70° C. to obtain a mixture II. Subsequently, the mixture II was added to the mixture I, and the resulting mixture was uniformly dispersed with a Henschel mixer to obtain a cosmetic base material. Water was added to this cosmetic base material, followed by heating to 60° C. and kneading with a kneader. The resulting slurry was degassed at 0.08 mPa for 5 minutes, was then packed in an inner tray (metal tray), was compression-molded at 5 kgf/cm², and was then dried in a dryer at 40° C. for 10 hours to obtain a face color sample.

### Evaluation

The resulting face color samples were evaluated as above, and it was demonstrated that the samples were evaluated to be “Excellent” in all of “usability”, “impact resistance”, “slurry state”, “moldability”, and “water repellency”.

### Example 18: Eye Shadow

Component: Blending ratio (% by mass)
1. Silicone-treated synthetic mica: 30.00
2. Synthetic phlogopite: 15.00
3. Silicone-treated talc: 10.00
4. Sericite: 5.90
5. Silicone-treated yellow iron oxide: 0.50
6. Silicone-treated black iron oxide: 0.10
7. Titanated mica A: 17.00
8. Titanated mica C: 3.00
9. Octyl stearate: 7.50
10. Vaseline: 2.00
11. Methyl polysiloxane: 7.00
12. Beeswax: 2.00

Water: 140 parts by mass based on 100 parts by mass of the total of the components 1 to 12.

The details of these components are the same as those described above, excepting the following components:

Synthetic phlogopite: PDM-9WB (manufactured by Topy Industries, Limited),

Titanated mica C: PRESTIGE Sparkling Silver (manufactured by Sudarshan Chemical Industries Limited),

Vaseline: Sunwhite P-150 (manufactured by Nikko Rica Corporation), and

Methyl polysiloxane: KF-96 A-100CS (manufactured by Shin-Etsu Chemical Co., Ltd.).

### Process for Preparation

Components 1 to 8 were uniformly dispersed with a Henschel mixer to obtain a mixture I. Separately, components 9 to 12 were mixed and were heated at 60° C. to 70° C. to obtain a mixture II. Subsequently, the mixture II was added to the mixture I, and the resulting mixture was uniformly dispersed with a Henschel mixer to obtain a cosmetic base material. Water was added to this cosmetic base material, followed by heating to 60° C. and kneading with a kneader. The resulting slurry was degassed at 0.08 mPa for 5 minutes, was then packed in an inner tray (metal tray), was compression-molded at 3 kgf/cm², and was then dried in a dryer at 40° C. for 10 hours to obtain an eye shadow sample.

### Evaluation

The resulting eye shadow samples were evaluated as above, and it was demonstrated that the samples were evaluated to be “Excellent” in all of “usability”, “impact resistance”, “slurry state”, “moldability”, and “water repellency”.

What is claimed is:

1. A method for producing a solid powder cosmetic material comprising:
preparing a slurry by mixing a cosmetic base material containing a powder ingredient and an oily ingredient with a dispersion medium containing water;
compression-molding and drying said slurry packed in a container, wherein
the oily ingredient contains an ester oil which is a liquid at 25° C., has a molecular weight of 300 to 600 and an IOB value of 0.5 or less, and beeswax; and
said powder ingredient contains a hydrophobic powder.

2. The method for producing a solid powder cosmetic material according to claim 1, wherein
the slurry is prepared by mixing the cosmetic base material with the dispersion medium at 60° C. to 95° C.

3. A solid powder cosmetic material comprising:
a powder ingredient; and
an oily ingredient, wherein
the oily ingredient contains an ester oil which is a liquid at 25° C., has a molecular weight of 300 to 600 and an IOB value of 0.5 or less, and beeswax; and
the powder ingredient contains a hydrophobic powder.

4. The solid powder cosmetic material according to claim 3 comprising;
the ester oil is contained in an amount of 5% to 25% by mass based on the total amount of the cosmetic material;
the beeswax is contained in an amount of 0.3% to 5% by mass based on the total amount of the cosmetic material; and
the hydrophobic powder is contained in an amount of 20% to 94.5% by mass based on the total amount of the cosmetic material.
